(19) United States
(12) Patent Application Publication
(10) Pub. No.: US 2010/0168257 A1
(43) Pub. Date: Jul. 1, 2010

(54) METAL-DOPED NICKEL OXIDES AS CATALYSTS FOR THE METHANATION OF CARBON MONOXIDE

(76) Inventors: Matthias Duisberg, Frankfurt (DE); Wilhelm F. Maier, St. Ingbert (DE); Michael Kraemer, Saarbruecken (DE)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/530,584

(22) PCT Filed: Mar. 11, 2008
(86) PCT No.: PCT/EP08/01903

§ 371 Date: Jan. 26, 2010

(30) Foreign Application Priority Data
Mar. 13, 2007 (EP) 07005139.6

(51) Int. Cl. C07C 1/02; B01J 23/889; B01J 23/89; B01J 21/04; B01J 23/78; B01J 21/06

(52) U.S. Cl. 518/715; 502/337; 502/327; 502/335; 502/328

(57) ABSTRACT

The invention relates to catalysts for the methanation of carbon monoxide, which comprise metal-doped nickel oxide of the composition (in mol %) (M1)ₐ(M2)_{b}Ni_{c}Oₓwhere a=0.1 to 5 mol %, b=3 to 20 mol % and c=100−(a+b) mol % and M1 comprises at least one metal of transition group VII or VIII of the PTE (=Periodic Table of the Elements) and M2 comprises at least one metal of transition group III or IV of the PTE. The catalysts can be used as pure catalysts or as supported catalysts, if appropriate applied as coatings to an inert support body. They display high conversion and high selectivity and are used in methanation processes of CO in hydrogen-containing gas mixtures, in particular in reformates for operation of fuel cells. The catalysts of the invention can be prepared by precipitation, impregnation, sol-gel methods, sintering processes or by powder synthesis.

The invention relates to metal-doped nickel oxide catalysts for the selective hydrogenation of carbon monoxide to methane (“methanation” of CO). Such catalysts are used, for example, for removing carbon monoxide from hydrogen-containing gas mixtures as are used as reformate gases in fuel cell technology. These catalysts can also be used for removing CO from synthesis gases for the synthesis of ammonia. The invention further relates to a process for methanation of carbon monoxide employing such metal-doped nickel oxide catalysts and to a method of manufacture the catalyst materials.

A focus of use of these catalysts is in the purification of reformate gases for fuel cells. Problems associated with the provision and storage of hydrogen continue to prevent the wide use of membrane fuel cells (polymer electrolyte membrane fuel cells, PEMFCs) for mobile, stationary and portable applications. For relatively small stationary systems used, for example, in the household energy sector, the production of hydrogen from liquid or gaseous energy carriers such as methanol or natural gas by means of steam reforming followed by a water gas shift reaction is a promising alternative. The reformate gas formed in this way contains hydrogen, carbon dioxide (CO₂) and water and also small amounts of carbon monoxide (CO). The latter acts as a poison for the anode of the fuel cell and has to be removed from the gas mixture by means of a further purification step. Apart from selective oxidation (“PROX”), methanation, i.e. the hydrogenation of CO to methane (CH₄), in particular, is a suitable method of reducing the concentration of CO in hydrogen-rich gas mixtures to contents below 100 ppm.

However, the simultaneous presence of carbon dioxide (CO₂) in the reformate gases places particular demands on the reaction conditions and on the catalyst. The objective is to remove the CO which acts as catalyst poison in the fuel cell from the reformate gas stream as completely as possible without at the same time converting the CO₂ which is present in a large excess into methane and thus reducing the proportion of hydrogen. The most important reactions (1) and (2) for methanation are shown below:

CO+3H₂==>CH₄+H₂O (1)

CO₂+4H₂==>CH₄+H₂O (2)

The undesirable reaction (2) consumes more hydrogen than the desired reaction (1). The small proportion of CO in the reformate gas (about 0.5% by volume) compared to the proportion of CO₂ (about 20% by volume) makes it clear that the selectivity is an important parameter for the quality of a methanation catalyst. In general, the selectivity is defined as

Selectivity: S=Conv(CO)/[Conv(CO)+Conv(CO₂)],

where the conversion Conv is defined as

Conversion (%) Conv=[n(feed gas)−n(product gas)/n(feed gas)]×100,

where n=number of moles or concentration.

In the present application, the temperature difference ΔT_{CO2/CO} which is defined as follows:

ΔT_{CO2/CO}=T₁₀(CO₂)−T₅₀(CO)

where

T₅₀(CO)=temperature at which 50% of the CO fed in is reacted

T₁₀(CO₂)=temperature at which 10% of the CO₂ fed in is reacted,

is employed as characteristic indicator for the selectivity of a methanation catalyst.

The greater the temperature difference ΔT_{CO2/CO}, the more selectively does the methanation catalyst operate, since the undesirable secondary reaction of methanation of CO₂ (2) then commences only at significantly higher temperatures than the desired methanation of CO (1). A higher hydrogen yield in the purification of the reformate is achieved as a result of the suppression of the methanation of CO₂ (2). This in turn results in higher total efficiencies and thus to improved economics of the hydrogen-operated fuel cell system.

Catalysts for the methanation of CO have been known for some time. In most cases, a nickel catalyst is used. Thus, CH 283697 discloses an industrial process for the catalytic methanation of carbon oxides in hydrogen-containing gas mixtures, in which a catalyst comprising nickel, magnesium oxide and kieselguhr is used.

U.S. Pat. No. 4,318,997 also describes a nickel-containing methanation catalyst.

However, catalysts containing noble metals are also known. S. Takenaka and coworkers have described supported Ni and Ru catalysts. Complete conversion of CO was able to be achieved by means of catalysts of the compositions 5% by weight Ru/ZrO₂ and 5% by weight Ru/TiO₂ at 250° C. (cf. S. Takenaka, T. Shimizu and Kiyoshi Otsuka, International Journal of Hydrogen Energy, 29, (2004), 1065-1073). However, the catalysts described have a narrow temperature range for selective methanation of CO. Above 513K (=240° C.), methane formation by methanation of CO₂ is significantly increased.

In WO 2006/079532, a Ru catalyst (2% by weight of Ru on TiO₂/SiO₂) is used for the selective methanation of CO.

WO 2007/025691 discloses bimetallic iron-nickel or iron-cobalt catalysts for methanation of carbon oxides.

The general problem with conventional methanation catalysts is the CO₂ which is simultaneously present in excess. While the hydrogenation of CO initially predominates at low temperatures, methanation of CO₂ occurs to an increased extent as soon as most of the CO has been reacted. The above-described Ru-containing materials are also expensive because of the high noble metal content.

It was therefore an object of the present invention to provide improved catalysts for the methanation of carbon monoxide (CO), which convert CO in a hydrogen-containing gas mixture which at the same time contains CO₂ into methane with high conversion and high selectivity. They should have a minimal reactivity towards CO₂ so that they suppress the consumption of further hydrogen (H₂) in the methanation reaction and thus give a high hydrogen yield. A further object of the present invention was to provide a method for producing such catalysts, a process for methanation of CO employing such catalysts and a method for their use.

This first object is achieved by provision of catalysts according to claim 1. The process for producing the catalysts, the methanation process employing such catalysts as well as their use is described in further claims.

It has been found that specific nickel oxides which contain various dopants can be used as catalysts for the methanation of CO and in this reaction display very good properties in respect of conversion and selectivity.

The invention provides a catalyst for the methanation of carbon monoxide in hydrogen-containing gas mixtures, which comprises metal-doped nickel oxide of the composition (in mol %)

(M1)ₐ(M2)_{b}Ni_{c}Oₓ

where a=0.1 to 5 mol %,
b=3 to 20 mol %
c=100−(a+b) mol %
   and M1 comprises at least one metal of transition group VII or VIII of the PTE (=Periodic Table of the Elements) and M2 comprises at least one metal of transition group III or IV of the PTE.

Here, M1 comprises at least one metal of the group manganese (Mn), rhenium (Re), iron (Fe), cobalt (Co), platinum (Pt), ruthenium (Ru), palladium (Pd), silver (Ag), gold (Au), rhodium (Rh), osmium (Os), iridium (Ir) and mixtures or alloys thereof.

Preferably, M1 comprises rhenium (Re), platinum (Pt), ruthenium (Ru), palladium (Pd), silver (Ag), gold (Au), rhodium (Rh), osmium (Os), iridium (Ir) and mixtures or alloys thereof.

M1 more preferably encompasses the group of noble metals, i.e. platinum (Pt), ruthenium (Ru), palladium (Pd), silver (Ag), gold (Au), rhodium (Rh), osmium (Os) or iridium (Ir), and mixtures or alloys thereof.

Most preferably, M1 encompasses the metals platinum (Pt) or rhenium (Re) and mixtures or alloys thereof.

Furthermore, M2 comprises at least one metal of the group scandium (Sc), yttrium (Y), lanthanum (La), titanium (Ti), zirconium (Zr) or hafnium (Hf) and mixtures or alloys thereof.

Preferably, M2 comprises at least one metal of transition group IV of the PTE, i.e. titanium (Ti), zirconium (Zr) or hafnium (Hf) and mixtures or alloys thereof.

The composition of the doped nickel oxide is reported in mol % based on the metals. The total of the metallic components a, b and c is 100 mol % (a+b+c=100 mol %). The index “x” in NiOₓ means that the actual, precise content of oxygen in the nickel oxide is not known or has not been examined in detail. The term “doped” in this context means an addition of at least two further metallic components in a total amount of from 0.5 to 25 mol %. Thus, for the compositions of the present invention the content of nickel oxide is in the range of 75 to 99.5 mol %.

Doped nickel oxides doped by the metals M1=platinum (Pt) and/or rhenium (Re) and also by the metals M2=hafnium (Hf), yttrium (Y) and/or zirconium (Zr) are preferred as catalysts. Examples of such preferred compositions are Re₂Hf₉Ni₈₉Oₓ, Pt_{0.6}Y₁₁Ni_{88.4}Oₓ or Re₂Zr₁₀Ni₈₈Oₓ.

Doped nickel oxides doped by the metal M1=rhenium (Re) and also by the metal M2=zirconium (Zr) are particularly preferred as catalysts. Examples of such particularly preferred compositions are Re₂Zr₁₀Ni₈₈Oₓ or Re₅Zr₅Ni₉₀Oₓ.

It has surprisingly been found that the metal-doped nickel oxides of the type (M1)ₐ(M2)_{b}Ni_{c}Oₓ give a significantly better conversion and a higher selectivity in the methanation of CO in the temperature range from 180 to 270° C., preferably in the temperature range from 180 to 250° C. and more preferably in the temperature range from 200 to 250° C., than do systems known from the literature. With these wide temperature ranges, the catalysts of the invention display a large operating window. At an operating temperature of 250° C., the CO conversions are typically >75%, preferably >80%.

The metal-doped nickel oxides of the invention can be used in pure form, i.e. as “pure catalysts”, in the form of pellets, spheres or powder. Depending on the application, it can be necessary to adjust the particle size, particle size distribution, specific surface area, bulk density or porosity of the catalyst formulation of the invention by variation of the production parameters or by means of additional process steps (for example calcination, milling, sieving, pelletization, etc.). The manufacturing steps necessary for this purpose are known to those skilled in the art. The catalyst can be obtained in the amorphous state or in the crystalline state.

However, the metal-doped nickel oxides can also be used in supported form. To produce a supported catalyst, the doped nickel oxide is applied as catalytically active component (“active phase”) to a suitable support material. Support materials which have been found to be useful are inorganic oxides such as aluminium oxide, silicon dioxide, titanium oxide, rare earth oxides (“RE oxides”) or mixed oxides thereof and also zeolites. To attain a very fine distribution of the catalytically active component on the support material, the support material should have at least a specific surface area (BET surface area, measured in accordance with DIN 66132) of more than 20 m²/g, preferably more than 50 m²/g. The amount of inorganic support material in the catalyst should be in the range from 1 to 99% by weight, preferably from 10 to 95% by weight (in each case based on the amount of metal-doped nickel oxide).

To effect thermal stabilization and/or as promoters, the catalysts of the invention can contain an inorganic oxide selected from the group consisting of boron oxide, bismuth oxide, gallium oxide, tin oxide, zinc oxide, oxides of the alkali metals and oxides of the alkaline earth metals and mixtures thereof in an amount of up to 20% by weight in addition to the active phase (i.e. in addition to the metal-doped nickel oxide), with the specified amount being based on the amount of the metal-doped nickel oxide. The stabilizers can be added during the production process, for example before gel formation, or afterwards.

Furthermore, the metal-doped nickel oxides of the invention can be applied either in pure form or in supported form (i.e. as supported catalyst, see above) as coating to inert support bodies. Such a catalyst will hereinafter also be referred to as a coated catalyst. Suitable support bodies are the monolithic honeycomb bodies made of ceramic or metal and having cell densities (number of flow channels per unit cross-sectional area) of more than 10 cm⁻² which are known from automobile exhaust gas purification. However, metal sheets, heat exchanger plates, open-celled ceramic or metallic foam bodies and irregularly shaped components can also be used as support bodies. For the purposes of the present invention, a support body is referred to as inert when the material of the support body does not participate or participates only insignificantly in the catalytic reaction. In general, these are bodies having a low specific surface area and a low porosity.

The present invention further relates to a production process for the metal-doped nickel oxide catalysts of the invention.

The catalysts of the invention can be produced by precipitation, impregnation, a sol-gel method, sintering processes or simple powder synthesis. A preferred method of production is the sol-gel method. Here, the respective starting salts (for example nickel nitrate, zirconyl nitrate or rhenium chloride) are firstly dissolved using alcoholic solvents and suitable complexing agents (sol production) and this solution is then aged, resulting in formation of the corresponding gel. The gel is dried and, if appropriate, calcined. The gel is generally dried in air at temperatures in the range from 20 to 150° C. Typical calcination temperatures are in the range from 200 to 500° C., preferably from 200 to 400° C., in air. The finished catalyst can subsequently be processed further.

To produce a supported catalyst, a high-surface-area support material (for example Al₂O₃ from SASOL having a specific surface area determined by the BET method of 130 m²/g) can be added to the reaction mixture in a particular amount before gel formation. After gel formation has occurred, the powder is separated off, dried and calcined. However, the support material can also be mixed with the active phase after production of the metal-doped nickel oxide.

To produce a coated catalyst body (“coated catalyst”), the finished catalyst powder (either in supported form or as pure powder), if appropriate together with stabilizers and/or promoters, is slurried in water and applied to a monolithic support body (a ceramic or metal). This coating suspension can, if appropriate, contain binders to improve adhesion. After coating, the monolith is subjected to thermal treatment. The catalyst loading of the monolith is in the range from 50 to 200 g/l. The catalyst is installed in an appropriate reactor for operation or testing.

The present invention further relates to a process for methanation of CO in hydrogen-containing gas mixtures by use of the catalyst materials described herein. The methanation process is conducted in suitable reactors in a temperature range from 180 to 270° C., preferably in a temperature range from 180 to 250° C. and more preferably in a temperature range from 200 to 250° C. The hydrogen-containing gas mixtures are generated in a fuel processor system (also called “reformer”) and typically comprise 0.1 to 5 vol. % CO, 10 to 25 vol. % CO₂, 40 to 70 vol. % hydrogen and balance nitrogen. Preferably, the hydrogen-containing gas mixtures comprise 0.1 to 2 vol. % CO, 10 to 25 vol. % CO₂, 40 to 70 vol. % hydrogen and balance nitrogen. Further process details are given in the Examples section (ref to “Examination of catalytic activity”).

### Examination of Catalytic Activity

The catalytic activity of the catalysts was tested on powder samples in a tube reactor. For this purpose, 100 mg of catalyst were introduced into a heatable glass tube. The conversion of the starting materials was determined as a function of temperature in the range from 160 to 340° C. A Ru/TiO₂ catalyst (cf. comparative example CE1) known from the literature was employed as reference catalyst. The temperature difference ΔT_{CO2/CO} (cf. introductory part) serves as characteristic parameter for the selectivity of a methanation catalyst.

### Examination of the Long-Term Stability

The assessment of the long-term stability was carried out in a flow reactor. A deactivation rate D_{R}=dU/dt in %/h is determined as measure of the long-term stability. To measure the long-term stability, the material is introduced into a reactor, with the catalysts being supported and applied to structured bodies (e.g. monoliths). The CO conversion in the product gas is determined at constant temperature over a period of 50 hours.

The following examples illustrate the invention without restricting its scope.

### EXAMPLES

### Example 1

Preparation of Re₂Hf₉Ni₈₉Oₓ

7.21 ml (94.17 mmol) of isopropanol and 2.229 ml (18 mmol) of 4-hydroxy-4-methyl-2-pentanone (from Aldrich) are placed in a 20 ml glass vessel while stirring. 5.34 ml of a 1M Ni(C₂H₅COO)₂ solution in methanol, 1.8 ml of 0.3M HfCl₄ (from Aldrich; in methanol) and 1.2 ml of a 0.1M ReCl₅ solution (from Aldrich; in methanol) are subsequently pipetted in. The brown-green solution is then stirred for 1 hour and subsequently aged open in a fume hood. This results in formation of a deep greenish brown, highly viscous, clear gel which is subsequently dried at 40° C. in a drying oven. Calcination of the gel is carried out at 350° C. This gives a black powder.

### Example 2

Preparation of Pt_{0.6}Y₁₁Ni_{88.4}Oₓ

8.42 ml (109.98 mmol) of isopropanol and 2.229 ml (18 mmol) of 4-hydroxy-4-methyl-2-pentanone (from Aldrich) are placed in a 20 ml glass vessel while stirring. 5.30 ml of a 1M Ni(C₂H₅COO)₂ solution in methanol, 2.2 ml of a 0.3M Y(NO₃)_{3×6}H₂O solution (from Aldrich; in methanol) and 0.36 ml of a 0.1M PtBr₄ solution (from Alpha Aesar; in isopropanol) are subsequently pipetted in. The brown-green solution is then stirred for 1 hour and subsequently aged open in a fume hood. This results in formation of a deep greenish brown, highly viscous, clear gel which is subsequently dried at 40° C. in a drying oven. Calcination of the clear, vitreous gel obtained is carried out at 350° C. in air. This yields a black-green powder.

### Example 3

Preparation of Re₂Zr₁₀Ni₈₈Oₓ

6.94 ml (90.65 mmol) of isopropanol and 2.229 ml (18 mmol) of 4-hydroxy-4-methyl-2-pentanone are placed in a 20 ml glass vessel while stirring. 5.28 ml of a 1M Ni(C₂H₅COO)₂ solution in methanol, 2 ml of a 0.3M ZrO(NO₃)₂ solution (from Johnson Matthey; in methanol) and 1.2 ml of a 0.1M ReCl₅ solution (likewise in methanol) are subsequently pipetted in. The brown-green solution is then stirred closed for 1 hour and subsequently aged open in a fume hood. This results in formation of a deep greenish brown, highly viscous, clear gel which is subsequently dried at 40° C. Calcination of the clear, vitreous gel obtained is carried out at 350° C. in air. This gives a deep green to black powder.

### Comparative Example (CE1)

### Production of Ru/TiO₂

500 mg (6.26 mmol) of titanium oxide (type P25, from Degussa; BET ˜120 m²/g) are slurried in water and admixed with 103.6 mg (0.096 mmol) of Ru(III) chloride solution (Ru content=19.3% by weight; from Umicore, Hanau). After addition of 20% strength NH₄CO₃ solution, the Ru is fixed on the support oxide. The product formed is evaporated to dryness and treated at 500° C. in a furnace. Composition: 4% by weight of Ru on TiO₂ (based on support material).

### Example 4

### Production of a Supported Catalyst

A catalyst having the composition described in Example 3 is prepared. However, a high-surface-area Al₂O₃ (from SASOL, BET: 130 m²/g) is added in a weight ratio of catalyst/support material of 1:4 with stirring before gel formation, with the proportions of solvent being adapted accordingly. The remaining working steps are carried out as described in Example 3. This gives a grey powder comprising 20% by weight of Re₂Zr₁₀Ni₈₈Oₓ (active phase) on 80% by weight of Al₂O₃ (support material).

### Example 5

### Production of Coated Support Bodies (Metal Sheets)

A powder as described in Example 3 or as described in Comparative Example 1 (CE1) is slurried in water and admixed with Al₂O₃ (from SASOL, BET: 130 m²/g) in a weight ratio of catalyst/support material of 1:2 (for CE1, in a weight ratio of 1:1). The slurry produced in this way is applied to metal sheets. The catalyst loading of the sheet is 50 g/m². After thermal treatment, the coated support body is introduced into an isothermal reactor. The catalysts are examined in a long-term test in which the deactivation rate is determined.

### Example 6

### Production of a Coated Support Body (Monolith)

The powder obtained in Example 4 is slurried in water and applied to a monolithic support body (cordierite ceramic, cell density=600 cells/inch²). The monolith is subsequently subjected to thermal treatment. The catalyst loading of the monolith is 130 g/l. The coated support body is introduced into a reactor; the deactivation rate is determined during operation at a constant temperature.

### Example 7

Preparation of Re₂Zr₁₀Ni₈₈Oₓ by Impregnation Method

Alternatively, the catalyst of Example 3 can be prepared by impregnation of NiO. In this method, 2.00 g (26.7 mmol) of nickel oxide (from Umicore) are impregnated with 10 ml of an aqueous solution containing 0.752 g (3.25 mmol) ZrO(NO₃)₂×H₂O (from Alfa-Aesar) and 0.236 g (0.65 mmol) ReCl₅ (from Aldrich). The material is dried and afterwards calcined at 350° C. in air. This yields a deep green to black powder.

### Examination of Catalytic Activity

The catalytic activity of the catalyst powders was tested in a tube reactor. For this purpose, 100 mg of catalyst were introduced into a heatable glass tube. The test conditions were:
Gas composition: 2 vol. % of CO, 15 vol. % of CO₂, 63 vol. % of H₂, 20 vol. % of N₂;
Gas flow: 125 ml/min
GHSV: ˜15 000 l/h

The conversion of the starting materials was determined as a function of temperature in the range from 160 to 340° C. The catalyst described in CE1 was employed as reference catalyst.

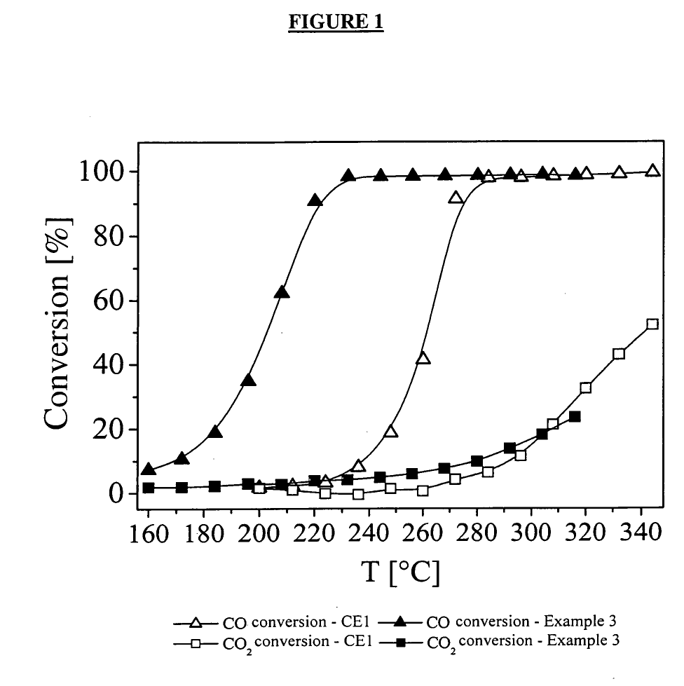

Conversions: The metal-doped nickel oxides according to the invention display significantly better conversions in the methanation of CO than does the reference catalyst CE1 even at temperatures of 220° C. (493K). As can be seen from FIG. 1, the catalyst according to the invention described in Example 3 (Re₂Zr₁₀Ni₈₈Oₓ) gives a CO conversion of 90% at 220° C. while the reference catalyst CE1 has virtually no activity (CO conversion<5%).

Selectivity: The greater the temperature difference ΔT=T₁₀CO₂−T₅₀CO, the more selective are the catalysts, since the undesirable secondary reaction of methanation of CO₂ then commences only at significantly higher temperatures than the desired reaction of CO. Table 1 summarizes the measured data. It can be seen that the temperature difference ΔT_{CO2/CO} (column 3) for the catalysts according to the invention is more than a factor of 2 above the value for the reference sample (CE1). This clearly demonstrates the improved selectivity of the catalysts of the invention.

TABLE 1
Measured data for selectivity

| | | T₅₀ CO | T₁₀ CO₂ | ΔT_{CO2/CO} |
|---|---|---|---|---|
| Example | Catalyst | (° C.) | (° C.) | (° C.) |
| CE1 | Ru/TiO₂ | 262 | 294 | 32 |
| 1 | Re₂Hf₉Ni₈₉Oₓ | 217 | 286 | 69 |
| 2 | Pt_{0.6}Y₁₁Ni_{88.4}Oₓ | 242 | 318 | 76 |
| 3 | Re₂Zr₁₀Ni₈₈Oₓ | 202 | 281 | 79 |

### Examination of Long-Term Stability

The testing of the long-term stability of the catalysts according to the invention was carried out in a flow reactor. A deactivation rate D_{R}=dU/dt (in %/h) is determined as a measure of the long-term stability. The conversion of CO in the product gas is determined at constant temperature over a period of 50 hours. The test conditions were:
Gas composition: 0.3 vol. % of CO, 15 vol. % of CO₂, 59.7 vol. % of H₂, 15 vol. % of H₂O, 10 vol. % of N₂.
GHSV: 10 000 l/h

The catalyst-coated support bodies, (the Re₂Zr₁₀Ni₈₈Oₓ catalyst prepared in Example 3 was used as active phase) produced as described in Example 5 (metal sheet) or as described in Example 6 (monolith) were introduced into an isothermal reactor and compared with the reference catalyst CE1 (applied to a metal sheet as support body as described in Example 5). The deactivation rates (D_{R}=dU/dt (in %/h)) shown in Table 2 were determined. It can be seen that the catalysts according to the invention display a significantly lower deactivation rate D_{R} than CE1.

TABLE 2
Deactivation rates in the long-term test

| Example | D_{R} (%/h) | Catalyst | Support body |
|---|---|---|---|
| CE1 | −0.125 | Ru/TiO₂ | metal sheet |
| 5 | −0.0275 | Re₂Zr₁₀Ni₈₈Oₓ | metal sheet |
| 6 | −0.020 | Re₂Zr₁₀Ni₈₈Oₓ | monolith |

1. Catalyst for the methanation of carbon monoxide in hydrogen-containing gas mixtures, which comprises metal-doped nickel oxide of the composition (in mol %)
(M1)ₐ(M2)_{b}Ni_{c}Oₓ
wherein
a=0.1 to 5 mol %,
b=3 to 20 mol %
c=100−(a+b) mol %
and
M1 comprises at least one metal of transition group VII or VIII of the PTE (=Periodic Table of the Elements) and M2 comprises at least one metal of transition group III or IV of the PTE.

2. Catalyst according to claim 1, wherein Ml encompasses the metals manganese (Mn), rhenium (Re), iron (Fe), cobalt (Co), platinum (Pt), ruthenium (Ru), palladium (Pd), silver (Ag), gold (Au), rhodium (Rh), osmium (Os), iridium (Ir) and mixtures or alloys thereof.

3. Catalyst according to claim 1, wherein M2 encompasses the metals scandium (Sc), yttrium (Y), lanthanum (La), titanium (Ti), zirconium (Zr) and hafnium (Hf) and mixtures or alloys thereof.

4. Catalyst according to claim 1, wherein
a=0.2 to 3 mol %
b=5 to 15 mol %.

5. Catalyst according to claim 1 which further comprises an inorganic support material having a specific surface area of more than 20 m²/g.

6. Catalyst according to claim 5, wherein the inorganic support material comprises aluminium oxide, silicon oxide, titanium oxide, rare earth oxides or mixed oxides thereof and zeolites.

7. Catalyst according to claim 5, wherein the proportion of the inorganic support material is in the range from 1 to 99% by weight, preferably from 10 to 95% by weight in each case based on the amount of the metal-doped nickel oxide.

8. Catalyst according to claim 1 which further comprises an inorganic oxide selected from the group consisting of boron oxide, bismuth oxide, gallium oxide, tin oxide, zinc oxide, oxides of the alkali metals and oxides of the alkaline earth metals in a concentration of up to 20% by weight based on the amount of the metal-doped nickel oxide.

9. Catalyst according to claim 1, wherein the catalyst has been applied to an inert support body.

10. Catalyst according to claim 9, wherein monolithic ceramic honeycomb bodies, metallic honeycomb bodies, metal sheets, heat exchanger plates, open-celled ceramic foam bodies, open-celled metallic foam bodies or irregularly shaped components are used as inert support body.

11. Process for producing the catalyst according to claim 1 by sol-gel processes.

12. Process according to claim 11, wherein an inorganic support material having a specific surface area (BET) of more than 20 m²/g is added before gel formation.

13. Process according to claim 11, wherein the gel is dried at temperatures in the range from 20 to 150° C.

14. Process according to claim 11, wherein the gel is calcined at temperatures in the range from 200 to 500° C.

15. A process for the methanation of CO in hydrogen-containing gas mixtures using the catalyst according to claim 1.

16. The process according to claim 15, wherein the hydrogen-containing gas mixture is brought into contact with the catalyst at temperatures in the range from 180 to 270° C.

17. The process according to claim 15, wherein carbon monoxide conversions above 75% are achieved at an operating temperature of 250° C.

18. The process according to claim 15, wherein the hydrogen-containing gas mixture is a reformate gas for operation of fuel cells.

19. Process for methanation of CO in hydrogen-containing gas mixtures, wherein a catalyst according to claim 1 is employed.
